(19) United States
(12) Patent Application Publication
(10) Pub. No.: US 2006/0253001 A1
(43) Pub. Date: Nov. 9, 2006

(54) Method for spatial disorientation identification countermeasures, and analysis

(75) Inventors: Ronald L. Small, Boulder, CO (US); John W. Keller, Boulder, CO (US); Alia M. Fisher, Broomfield, CO (US); Christopher D. Wickens, Boulder, CO (US)

Correspondence Address: LATHROP & GAGE LC, 4845 PEARL EAST CIRCLE, SUITE 300, BOULDER, CO, 80301, US

(73) Assignee: Micro Analysis and Design, Inc.

(21) Appl. No.: 11/203,718

(22) Filed: Aug. 15, 2005

Related U.S. Application Data
(60) Provisional application No. 60/678,919, filed on May 6, 2005.

(51) Int. Cl. A61B 5/00

(52) U.S. Cl. 600/300

(57) ABSTRACT

Provided is a spatial disorientation (SD) system and method for detecting, analyzing and responding to an SD event. An input routine includes receiving environment data and/or data from a subject within the environment, e.g., true position and orientation of the environment and/or subject. A vestibular attitude calculator computes perceived subject attitude for environment data elements, and may compute a Washout value. A vestibular illusion routine calculates the probability of a vestibular illusion. A threshold adjustment routine adjusts Washout and vestibular thresholds based on subject preference data. Probability and type of an SD event is determined by evaluating the received and computed data. Sensory countermeasures may be implemented responsive to the probability of an SD event. An output routine provides true position and orientation of the environment and perceived subject attitude via an output device; such information may be recorded for post-hoc review, a method of which is also provided.

### RELATED APPLICATION

This application claims priority of U.S. Provisional Application Ser. No. 60/678,919, filed on May 6, 2005, herein incorporated by reference.

### GOVERNMENT RIGHTS

The U.S. Government has a paid-up license in this invention and the right in limited circumstances to require the patent owner to license others on reasonable terms as provided for by the terms of Contract FA8640-04-C-6457, awarded by USAF/AFMC, Air Force Research Laboratory, 2310 Eighth Street, Building 167, Wright-Patterson AFB, Ohio 45433-7801.

### FIELD OF THE INVENTION

The invention relates generally to the field of human spatial disorientation (SD). It addresses the problems of identifying, correcting, and analyzing SD episodes as experienced by human subjects in environments where spatial disorientation may occur, such as, for example, an aircraft pilot in an aircraft.

### BACKGROUND

Spatial disorientation (SD) is the mistaken perception of a person's position, attitude and motion relative to the earth or significant objects visible to the person, such as, for example, mountains, trees, buildings or the like.

Spatial disorientation is a normal human response to accelerations in flight, and has been recognized since the early days of flight. The cost of SD to the U.S. military is over $300 million per year, with comparable costs to U.S. civil aviation. Without question, SD contributes more to the cause of aircraft accidents (civilian as well as military) than any other physiological problem in flight. Regardless of flight time experience, all aircrew members are subject to disorientation.

Despite significantly increased research over the past decade, the rate of accidents caused by SD has not decreased. With few exceptions, the most recent research emphases have been limited to understanding the physiology of SD. The new knowledge gained by such research has not been translated into tools (e.g., training, displays, automation) that help pilots avoid SD and minimize its effects if it does occur. Although a very common experience for pilots in aircraft, SD events and their associated problems may also be experienced by boat operators, divers, astronauts, firefighters and other persons in environments where visual cues may or may not agree with the perceived feelings of motion. Simply stated, if a person is in an environment with low visibility and impaired attitude awareness, there is an elevated chance they will experience an SD event.

There are three types of SD, Type 1—unrecognized, Type 2—recognized and Type 3—incapacitating, each of which is most commonly referred to with respect to aircraft pilots. With Type 1, the pilot is not aware of critical control or flight parameters of the aircraft, and therefore may control the aircraft with erroneous assumptions. With Type 2, the pilot perceives a problem (resulting from SD) but fails to recognize it as SD. Typically the pilot will believe that the aircraft instruments are malfunctioning. With Type 3, the pilot experiences such an overwhelming sensation of movement that he or she can not reorient himself or herself by using visual cues or the aircraft instruments.

A human being's perception of motion is a result of the vestibular system, otherwise known as the inner ear. The vestibular system (organ of equilibrium), consists of two structures—1) the semicircular canals, which detect changes in angular acceleration; and 2) the otolith organs (utricle and saccule), which detect changes in linear acceleration and gravity.

The semicircular canals are three half-circular, interconnected tubes in three planes perpendicular to each other. Each plane corresponds generally to rolling, pitching or yawing motions. Although there are two systems (one for each ear), they are collectively in operation as one system. Each canal is filled with a fluid called endolymph. A motion sensor is provided in the form of cupula (a gelatinous structure) and hairs extending from hair cells below the cupula; the ends of the hairs are embedded in the cupula. The cupula and the hairs move as the fluid inside the canal moves in response to an angular acceleration.

The otolith organs, the saccule and utricle, are located in each ear and are set at right angles to each other. The utricle detects changes in linear acceleration in the horizontal plane, while the saccule detects gravity changes in the vertical plane. Inertial forces resulting from linear accelerations cannot be distinguished from the force of gravity.

As the issue of SD can lead to loss of life and the destruction of property, several prior art methods have been developed in various efforts to address SD with respect to aircraft pilots. This art includes flight simulation devices such as described in U.S. Pat. No. 4,710,128 to Wachsmuth et al., which provides a controlled environment for creating SD events. However, certain SD events arise from conditions below human perception thresholds.

Also in the related art is U.S. Pat. No. 5,285,685 to Chelette, which provides a method and apparatus for communicating perceived attitude information from a test subject. Again, although perhaps beneficial for some instances of SD, there are forms of SD which are below perception and others that are so overwhelming that the subject loses all perception of perceived attitude.

U.S. Pat. No. 5,629,848 to Repperger et al., is focused upon an SD detector system capable of warning a pilot of potentially disorienting flight conditions in response to a Kalman filter modeling of human response characteristics. More specifically, a Kalman apparatus produces a state estimate of both the true position and orientation, as well as the pilot's perceived position and orientation of the aircraft.

The Repperger system is based in part on both the otolith and semicircular canal responses of human physiology. The Repperger system is an on-board only system, active only during flight, and its function is to determine only when an SD event is or is not occurring. When an error of sufficient magnitude occurs between the Kalman filter's true value and perceived estimate, an SD event is deemed to be occurring and a visual warning is provided to the pilot.

Repperger does not consider the developing probability of the SD event or the type of SD event. In addition, Repperger does not provide for post-event analysis and/or comparison to other similar events. Further still, Repperger does not consider either a range of warnings or different methods and/or types of delivery selected for the best chance of reaching the subject pilot and helping him or her overcome the SD event.

Hence, there is a need for a spatial disorientation identification method and system that overcomes one or more of the technical drawbacks identified above.

### SUMMARY

The present disclosure advances the art by providing a system and method for spatial disorientation identification, countermeasures and analysis.

In particular, and by way of example only, according to an embodiment, provided is a computer-readable medium on which is stored a computer program for detecting, analyzing and responding to a spatial disorientation event. The computer program includes an input routine operatively associated with an input device for receiving real time data, recorded data, subject preference information or combinations thereof. The data include environment data from an environment, the environment data including true position and orientation of the environment, and subject data from a subject within the environment. A vestibular attitude calculator routine computes the perceived subject attitude of the subject within the environment based on the environment data and subject data. The vestibular attitude calculator includes a Washout routine to calculate a Washout value; a vestibular illusion routine to calculate the probability of a vestibular illusion; a threshold adjustment routine permitting adjustment of Washout thresholds and vestibular thresholds based on provided subject preference information; a countermeasure routine operating in response to the Washout value and the probability of a vestibular illusion, and an output routine operatively associated with an output device to provide the true position and orientation of the environment and the perceived subject attitude.

In an alternative embodiment, provided is a method for analyzing a spatial disorientation event post-hoc, including: collecting environment data elements recorded from an environment; collecting subject data recorded from a subject within the environment; calculating perceived subject attitude of the subject within the environment for each environment data element; evaluating the environment data, the subject data and the perceived subject attitude to determine the probability of a spatial disorientation event, and reporting the probability of the spatial disorientation event.

In yet another alternative embodiment, provided is a method for combating spatial disorientation, including: collecting real time environment data from an environment; collecting real time subject data from a subject within the environment; calculating perceived subject attitude of the subject within the environment for each environment data element and predicting Washout; evaluating the environment data, the subject data, the perceived subject attitude and Washout to determine the probability of a spatial disorientation event and the type of spatial disorientation event; implementing, in response to the probability of a spatial disorientation event, at least one countermeasure; and recording the environmental data and subject data as event data for post-hoc review. The environment data include true position and orientation of the environment; the Washout evaluated as a non-linear element, and the countermeasure is selectively chosen from a group of multi sensory countermeasures and countermeasure actions based on the environment data and spatial disorientation probability.

### BRIEF DESCRIPTION OF THE DRAWINGS

FIG. 1 shows a high level block diagram for a spatial disorientation identification system in accordance with an embodiment;

FIG. 2 is a graph illustrating the non-linear property of Washout;

FIG. 3 is a graph illustrating the Leans illusion model according to an embodiment;

FIG. 4 is a graph illustrating the Coriolis illusion model according to an embodiment;

FIG. 5 is a graph illustrating the Graveyard Spiral illusion model according to an embodiment;

FIG. 6 is a high level flowchart illustrating spatial disorientation detection in real time with recordation for post hoc review according to an embodiment; and

FIG. 7 is a high level flowchart illustrating spatial disorientation detection in post hoc review.

### DETAILED DESCRIPTION

Before proceeding with the detailed description, it is to be appreciated that the present teaching is by way of example, not by limitation. The concepts herein are not limited to use or application with a specific type of system or method for combating spatial disorientation. Thus, although the instrumentalities described herein are for the convenience of explanation shown and described with respect to exemplary embodiments, it will be appreciated that the principles herein may be equally applied in other types of methods and systems for detecting and combating spatial disorientation.

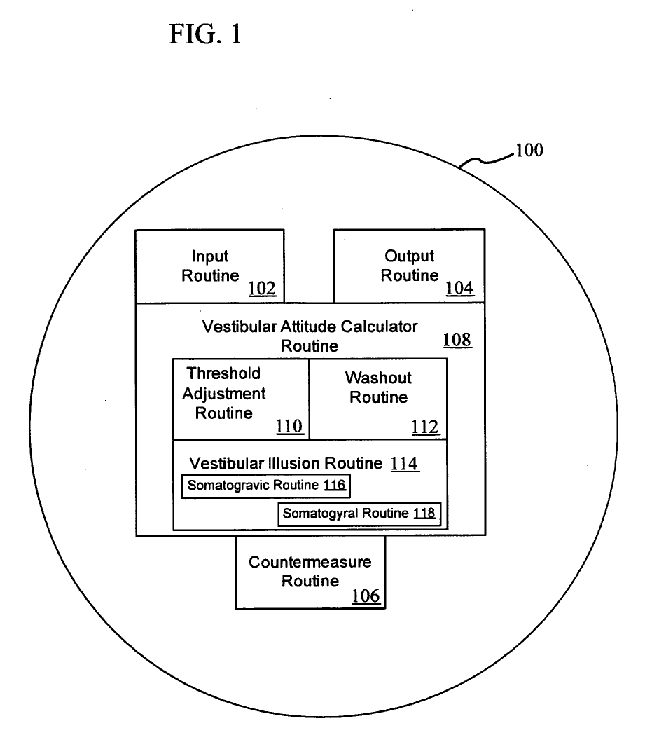

FIG. 1 is a high level block diagram of the computer program architecture of a spatial disorientation (SD) system 100 in accordance with at least one embodiment. SD system 100 may be implemented on a computer having typical computer components, such as a processor, memory, storage devices and input and output devices. During operation, SD system 100 may be maintained in active memory for enhanced speed and efficiency. In addition, in at least one embodiment, SD system 100 may be operated on a computer network and may utilize distributed resources.

To further assist in the following descriptions and discussion, the following defined terms are provided.

“Environment”—An aircraft, boat, vehicle or other setting which may provide conditions for an SD event to occur. In at least one embodiment, the Environment is an aircraft.

“Subject”—A person within the environment subject to the possibility of experiencing an SD event. In at least one embodiment, the Subject is an aircraft pilot.

“Countermeasure”—An action taken to prevent, minimize, or compensate for an SD event.

“Washout”—The diminishing capacity to appreciate continual motion at a constant rate.

“Threshold”—The level below which accelerations or motions are not sensed by the human vestibular system.

Threshold and Washout are significant factors in the onset of SD events. Within the relevant field of art, early research suggested the typical Threshold value for all axes could be Mulder's constant, two degrees per second. If a person experiences an acceleration of 1°/sec² for one second, he or she will probably not sense that acceleration because the product (1°/sec) is below Mulder's constant. If the same acceleration occurs for three seconds however, it will likely be detected because the product (3°/sec) exceeds Mulder's constant. It should also be recognized that even a large acceleration of 10°/sec² is unlikely to be felt if the duration is less than 0.2 seconds.

Mulder's constant, although helpful for general purposes has now been refined by Stapleford to reflect that the Threshold values are not uniform for all axes. As refined by Stapleford the general Threshold values are 3.2°/sec for roll, 2.6°/sec for pitch and 1.1°/sec for yaw. It is to be appreciated that acceleration may be linear acceleration and or rotational acceleration, and Washout can occur in either sense.

It is further important to note that not all humans have identical thresholds, however Mulder's constant is a good generalization. It is also important to note that not all acceleration durations above Mulder's constant will be sensed. Distractions, fatigue and other physiological reasons may exist to make the person oblivious to accelerations and/or durations that exceed Mulder's constant.

With respect to Washout, when one or more semicircular canals of the inner ear are put into motion, the fluid within the canal lags behind the accelerated canal walls. Lag of the fluid is sensed by hairs of the cupula and the brain interprets the movement of the hairs as motion in a direction. If motion continues at a constant rate for several seconds or longer, the fluid in the canals catches up with the canal walls and the brain receives the false impression that the turning has stopped, thus Washout has occurred.

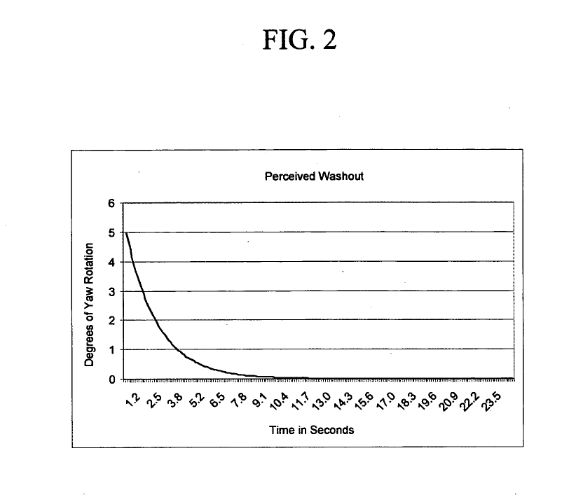

It is important to note that Washout is non-linear. More specifically, FIG. 2 shows an exponential decay curve that represents the change in perceived rotation over time. In the exemplary case the subject is a pilot who has accelerated into a turn and reached a 5°/sec yaw rotation. At this point, the turn is held such that the rate of rotation is close to constant (i.e., there are no above-threshold accelerations for a period of time). After 2.5 seconds, the sense of rotation is down to about 2°/sec. With respect to the true 5°/sec yaw rotation, this is a 3°/sec difference between actual and perceived rotation, or a Washout of about 60%. Although described with respect to yaw, Washout may occur for pitch or roll, as well.

As is further set forth and described below, SD system 100 is used to identify and combat spatial disorientation in accordance with the following primary, but not exclusive, tenets:
First—the SD system 100 acts to alert the subject with a countermeasure based on the probability of an SD event occurring, and the greater the probability, the more intense the countermeasure.
Second—in determining the probability of an SD event, the SD system 100 accounts for Washout of the subject, a calculation that in at least one embodiment is non-linear.
Third—the selectable countermeasures are multi sensory.
Fourth—the SD system 100 is capable of identifying a predicted SD event as a vestibular illusion (e.g., somatogravic or somatogyral illusion), and even more specifically, identifying the specific type of illusion.
Fifth—the SD system 100 permits both real time SD event determination and post-hoc analysis. With respect to aircraft flight for example, post hoc review permits researchers to simulate flight patterns and identify commonalities regarding SD events, thus permitting enhanced prediction of future SD events and evaluation of countermeasures applied to SD events.

Returning to FIG. 1, in at least one high level embodiment, SD system 100 includes an input routine 102, a countermeasure routine 106 and an output routine 104, each operably associated with a Vestibular Attitude Calculator (VAC) routine 108. The VAC routine 108 further includes a threshold adjustment routine 110, a Washout routine 112 and a vestibular illusion routine 114.

The input routine 102 is operatively associated with at least one input device for receiving real time data, recorded data, subject preference information and/or combinations thereof. The real time or recorded data include environment data from an environment (e.g., an aircraft), including true position and orientation.

The real time or recorded data also include subject data from the subject within the environment. In at least one embodiment the received subject data include vestibular Threshold values and Washout timing values. Minimum rotation threshold values may also be provided. In addition, the Threshold and Washout values may be independently defined for each axis of rotation (yaw, pitch, roll), based on Mulder's constant, Stapleford values, or individual subject preferences. Moreover, the subject may provide data to modify the SD event illusion parameters so as to increase the effectiveness of SD system 100 in determining the probability of an SD event. Subject data may also include information such as the subject's name, date and time.

In addition, in at least one embodiment, subject data include information indicating the true position and orientation of the subject's head. For example, the commercially available helmets worn by fighter pilots typically include devices such as Polhemus motion tracking system, accelerometers, micro-electrical mechanical devices, or other devices that accurately measure and report the roll, pitch and yaw angles of the pilot's head. Private pilots, boat operators, divers, firefighters, astronauts or other subjects in other environments may be easily fitted with commercially available accelerometers or other micro-electrical mechanical devices to provide similar roll, pitch and yaw angles of the subject's head. It is to be understood and appreciated that the pitch, yaw and roll axes of the subjects head (i.e., the pilot) correspond to the same pitch, yaw and roll axes of the environment (i.e., the aircraft). Further still, subject data may also include data from the controls operable by the subject, e.g., flight stick and foot pedals.

Moreover, in at least one embodiment, the real time or recorded subject data are gathered from at least one device worn by the subject within the environment. In at least one alternative embodiment, the real time or recorded subject data are gathered from environment controls operable by the subject within the environment. In further addition, in at least one embodiment, the real time or recorded data also include external world data, including for example time of day, visibility and noise level.

The output routine 104 is operatively associated with at least one output device to provide the true position and orientation of the environment and the perceived subject attitude. Such output data may be stored for later use or post hoc review, and/or made immediately available to the subject within the environment or a remote operator of the SD system 100. In at least one embodiment, the output routine 104 is coupled to a display and provides VCR-like controls (e.g., play, pause, stop, etc . . . ), an attitude indicator and 3D animation of actual vs. perceived environment attitude.

The countermeasure routine 106 is operable to provide a multisensory approach to preventing, minimizing or compensating for subject SD. More specifically, the countermeasure routine 106 is operable to initiate a range of different countermeasures including visual, auditory, olfactory and tactile actions. As the probability of an SD event increases, the countermeasure routine 106 is also operable to increase the implemented countermeasure from cautionary (e.g., flashing a warning or sounding an alarm) to emergency (e.g., engaging autopilot or ejecting the pilot).

The Vestibular Attitude Calculator (VAC) routine 108 is operable to perform all calculations associated with the vestibular system including Threshold assessment of acceleration in each axis, vestibular Washout in each axis and perceived attitude deltas. This is accomplished in at least one embodiment through the Threshold adjustment routine 110, the Washout routine 112 and the vestibular illusion routine 114. In at least one embodiment the vestibular illusion routine includes a somatogravic routine 116 (predicting illusions caused by change in linear accelerations and decelerations or gravity that affect the otolith organs), and a somatogyral routine 118 (predicting illusions caused by angular accelerations and decelerations stimulating the semicircular canals).

So as to effectively and advantageously initiate the most appropriate countermeasure for a perceived SD event, SD system 100 is not only capable of determining the probability of an SD event occurring, but in at least one embodiment is also capable of selectively identifying at least three types of SD illusions, namely, the Leans illusion, the Coriolis illusion, and the Graveyard Spiral illusion. The probabilistic determination that a Leans, Coriolis or Graveyard Spiral illusion is occurring is based on time sequence modeling.

The Leans Illusion

The Leans illusion is one of the most common vestibular based disorientations, and is primarily associated with the erroneous perception of changes in bank angle. The Leans results from a subject's failure to detect angular roll or banking motion. During continuous straight and level motion, the subject will correctly perceive that he or she is straight and level. However, if a bank is entered slowly (below Threshold) or maintained for a prolonged time, the fluid in the semicircular canals of the ear will stabilize and Washout will occur. If the subject is quickly returned to straight and level, the motion of the fluid in the semicircular canals will give the sensation that the subject is banking in the opposite direction, and the subject will have a tendency to lean back towards the original bank orientation as an attitude erroneously perceived to be straight and level.

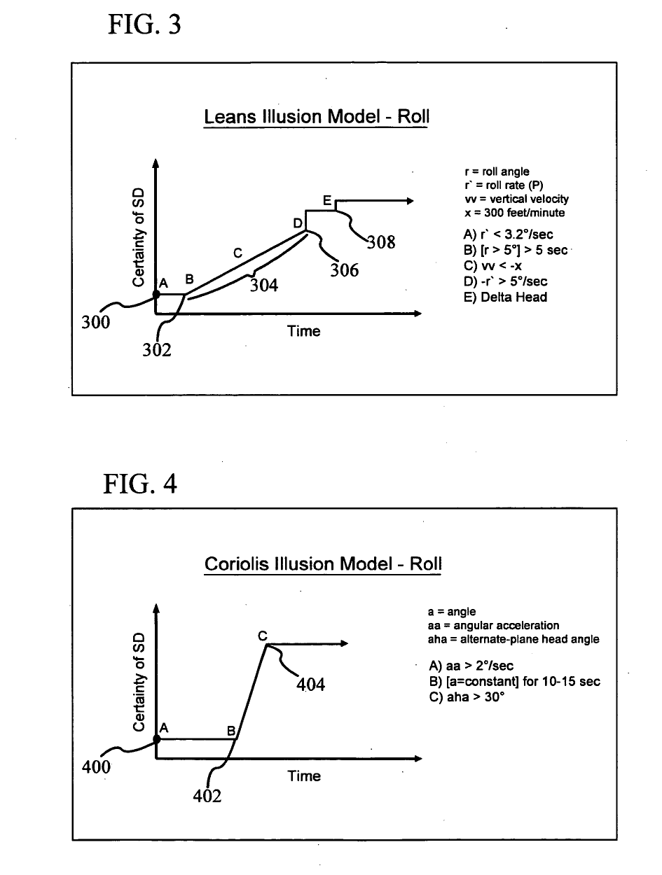

VAC routine 108 models the Leans illusion as a timed sequence of events with the probability of assessment of the disorientation increasing with each successive event. As indicated in FIG. 3, the first event 300 is the initiation of a roll at a rate below the vestibular threshold. Research investigating response to aircraft movements suggests that in at least one embodiment wherein the environment is an aircraft and the subject is a pilot, the default vestibular threshold values are 3.2°/sec for roll, 2.6°/sec for pitch and 1.1°/sec for yaw. The threshold adjustment routine permits these values to be adjusted for individual subjects. As shown in FIG. 3, the model is for the roll axis; however additional models exist for pitch and yaw as well.

The second event, 302 is a roll angle of greater than 5 degrees that lasts longer than 5 seconds. When the two events occur in sequence, it is possible that the pilot has not noticed the ensuing roll angle and as such that there is a difference between the subject's perceived attitude and the true attitude of the environment. As such the method indicates a possibility of an SD event, but only at a very low confidence level.

The third event 304 is the loss of altitude as measured by negative vertical velocity. In the model illustrated this is three hundred feet per minute, although this value is also adjustable. If this event follows events 300 and 302 in sequence, it is possible that the subject has not noticed the loss of altitude and the method represents an increased confidence in the assessment of the Leans.

The fourth event 306 is a roll well above the vestibular threshold in the opposite direction from that of the first event. If this occurs in sequence following events 300, 302 and 304, it is possible that the subject has noticed the roll angle and has quickly corrected back towards level. When this occurs, the subject's vestibular system registers a roll in the opposite direction, again resulting in a difference between the perceived attitude and the actual attitude of the environment. At this point the method represents a high level of SD event certainty.

The fifth and final event 308 is the tilt of the pilot's head opposite the perceived roll angle. If this occurs following events 300, 302, 304 and 306, it is likely that the pilot is experiencing the Leans illusion and the model represents an even higher level of SD event certainty.

The Coriolis Illusion

The Coriolis illusion is the most dangerous of all vestibular illusions, causing overwhelming disorientation. The Coriolis illusion involves the simultaneous stimulation of two semicircular canals. It occurs when a subject experiencing a prolonged turn makes a sudden head motion in a different geometrical plane from the plane of the turn (such as by suddenly tilting the head forward or backwards).

When in a prolonged turn, the semicircular canal corresponding to the yaw axis will equalize. The endolymph fluid in the semicircular canals no longer deviates, or moves the cupula; thus the hairs of the cupula are not bent. If the person initiates a head movement in a geometrical plane other than that of the turn, the yaw axis semicircular canal is moved from the plane of rotation to a new plane of non-rotation. The fluid then slows in that canal, resulting in a sensation of a turn in the opposite direction of the original turn. Simultaneously, the other two canals are brought within a plane of rotation, the fluid stimulation in those other two canals creates the perception of motion in three different planes of rotation—yaw, pitch and roll—all at the same time.

When a person's head is suddenly tilted or moved in one direction or another, the brain usually is able to compensate very quickly with involuntary eye movement in the opposite direction. It is this behavior that permits a person to maintain visual fixation upon an object while his or her body is being jostled about. This typically helpful involuntary behavior can be a serious problem when experiencing the Coriolis illusion, for the subject may experience very rapid involuntary eye movement that further leads to feelings of disorientation. In addition, the involuntary eye motion often makes it impossible for the subject to visually perceive his or her actual orientation either from the horizon, instruments, or other objects. Visual warning systems or indicators are also effectively mooted during an episode of the Coriolis illusion.

Based on this description, an embodiment of the method employs a model of the Coriolis illusion as a time sequence of events that include the Washout effect on the vestibular system associated with the prolonged constant-rate turn. While the Coriolis illusion primarily occurs when Washout occurs in the yaw plane, the model also accounts for the potential for Washout in both pitch and roll.

More specifically, as shown in FIG. 4, the first event 400 is an above threshold acceleration in any of the three planes. The second event 402 requires that the acceleration from the first event 400 result in a sustained rate of rotation sufficient to induce Washout. Where the method is embodied in the SD system 100, the Washout level is calculated by the VAC routine 108. In at least one embodiment, the level of Washout achieved in the second event 402 is at least 50%. As shown in FIG. 4, the model is for the roll axis; however additional models exist for pitch and yaw as well.

The third event 404 is a large movement of the head in a plane other than the plane of acceleration while the conditions of the first and second events 400, 402 are maintained. If this large motion of the head is detected, the probability of a Coriolis illusion occurring as an SD event is quite high. Although it is ideal to perceive the true orientation of the subject's head, such as when the subject is wearing a motion sensing helmet, the Coriolis illusion can also be predicted by observing awkward and/or radically inappropriate operations of the subject's controls and/or the attitude of the environment itself following the onset of the first and second events 400, 402.

The Graveyard Spiral Illusion

The Graveyard Spiral illusion is yet another somatogyral illusion commonly associated with fixed-wing aircraft. For example, the subject enters a turn (constant rotation) and remains in that turn for a sufficient time to induce Washout. This is then followed by correcting back to a straight and level path. The equalized fluid in the semicircular canals of the subject now moves in the opposite direction inducing a strong sensation of rotation in the opposite direction to the initial turn. To correct for this illusion the subject will then control the environment to correct for the perceived turn, an action that actually puts the environment back into the original rotation; however, the subject feels that he or she is traveling straight.

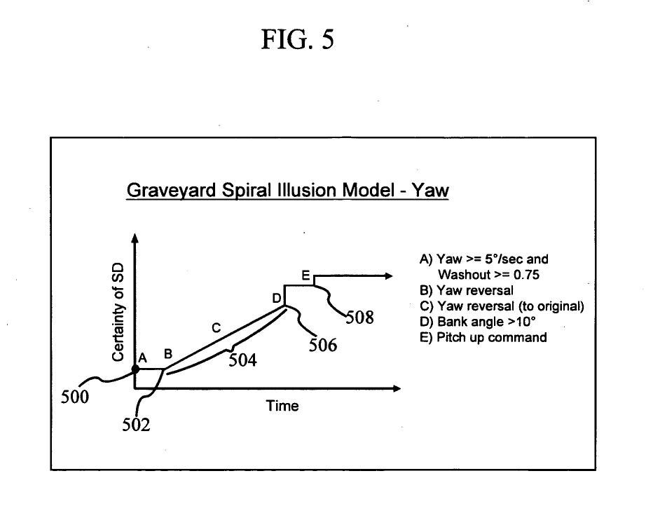

Based on this description, an embodiment of the method employs a model of the Graveyard Spiral illusion as a time sequence of events of rotations in the yaw axis. As shown in FIG. 5, the first event 500 is sustained rotation sufficient to result in a high level of Washout (e.g., 75%) in the yaw axis. This is then followed by the second event 502, an above-threshold acceleration in the opposite direction—the standard action to end the original turn. The third action 504 is an above threshold yaw acceleration in the original direction prior to the subject recovering from the Washout effect, an event indicating a reasonable probability of an SD event. The model of FIG. 5 is for the yaw axis; however additional models exist for pitch and roll as well.

In the case where the environment is an aircraft, the fourth event 506 is negative vertical velocity while banked in the unperceived turn. Negative vertical velocity is the loss of altitude due to the loss of lift while in the unperceived turn. Such an event raises the probability of the SD event to yet a higher level of confidence. A fifth event 508 is the pitch up of the control inputs while banked, causing an increasingly tighter downward spiral.

This fifth event is due to the subject (e.g., pilot) feeling no rotation but being aware of the loss in altitude and pulling back on the stick. Were he or she in the perceived straight and level flight, this action would result in the environment (e.g., aircraft) climbing. As he or she is actually in a banked turn, pulling back on the stick places the environment (e.g., aircraft) into a tighter spiraling descent. This fifth event raises the probability of the SD event yet higher.

To advantageously predict with increasing confidence levels of the onset of vestibular illusions, in at least one embodiment, a state table is employed. More specifically, the VAC routine 108 of SD system 100 is associated with a state table. The state table maintains the state of the subject, the environment and the external world (if the method or system is collecting external world data), and permits the vestibular illusion routine (e.g., vestibular illusion routine 114) to track the progression of events, such as those described above with respect to the Leans, Coriolis and Graveyard Spiral illusions. The state table may also be used to track the increasing confidence level that an SD event is occurring, and to determine whether the subject has responded to an initiated countermeasure.

Whereas prior systems, such as U.S. Pat. No. 5,269,848 to Repperger, use a specific threshold of difference between actual attitude and a Kalman filter model of attitude to determine whether an SD event is or is not occurring, the SD system 100 and disclosed method provides a sliding scale of confidence in predicting whether or not an SD event is occurring. Such a sliding scale of confidence prediction permits appropriate countermeasures to be implemented before a true crisis might develop, and for the implemented countermeasures to increase from cautionary to emergency as the probability of the SD event increases.

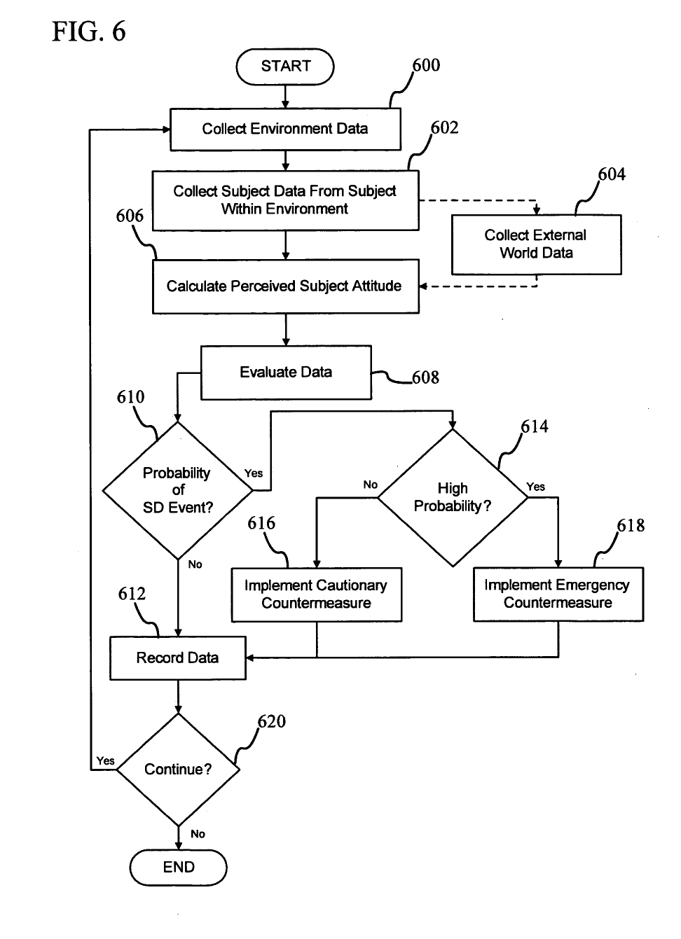

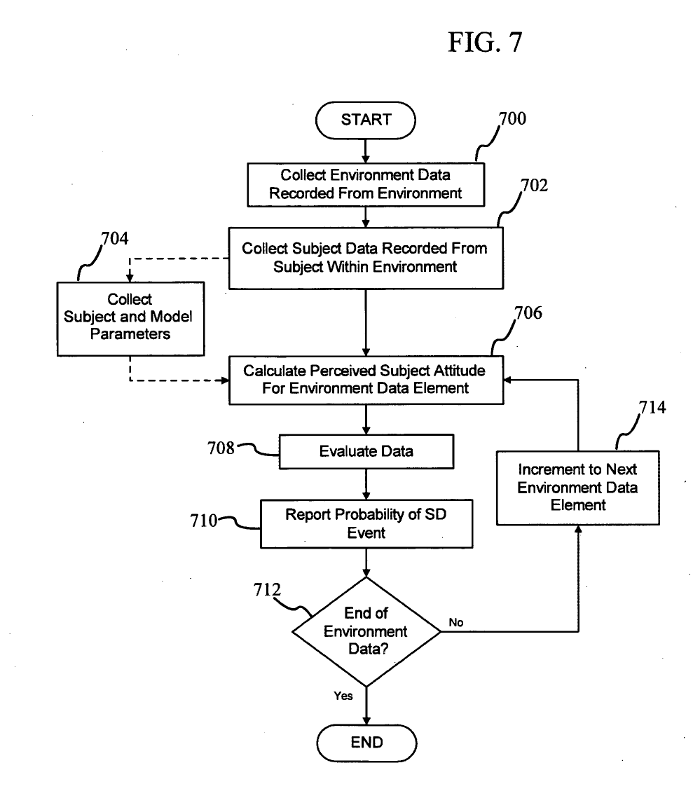

SD system 100 is advantageously operational either as an onboard system for combating spatial disorientation or as a post-hoc analysis tool. FIG. 6 illustrates a general high level flow diagram of an embodiment of a method applied onboard an environment, such as for example an aircraft, and FIG. 7 illustrates a general high level flow diagram of an embodiment of a method applied for post hoc review. It is understood and appreciated that the described processes need not be performed in the order in which they are herein described, but that this description is merely exemplary of at least one preferred method.

As illustrated in the real time detection environment of FIG. 6, in at least one embodiment, the method commences with the real time collection of environment data, block 600. The environment data include the true position and orientation of the environment.

Real time data are also collected from the subject within the environment, block 602. In at least one embodiment, the subject data include the true position and orientation of the subject, and more specifically the subject's head, within the environment. Subject modifiable parameters such as timing and Washout adjustments are in at least one embodiment established before activity in the environment (e.g., flight) commences.

In at least one embodiment, optional external world data are also collected as indicated by optional block 604. Such external world data include time of day and visibility, and may include other information such as, for example, level of ambient noise or temperature. For example, a photo sensor may be used to determine whether it is bright and sunny, overcast or dark.

It is understood and appreciated that environment data, subject data and external world data are collected at regular time intervals. Such time intervals may be on the order of seconds or fractions of seconds such that the data appear as a continuous stream of data elements. Such data may also be collected at different intervals with the interval decreasing as the onset of events indicates the developing possibility of an SD event.

With respect to situations where the environment is an aircraft and the subject is a pilot, in at least one embodiment, the environment data are gathered from one location, such as the aircraft's center of gravity, and the subject data are received from a different location, such as where the pilot is sitting. As the pilot is typically located some distance away from the aircraft's center of gravity, the effects of roll, pitch and yaw upon the pilot are different from their effects upon the center of gravity of the aircraft.

The perceived attitude of the subject is then calculated for each increment of time for which there is collected environment data, block 606. In addition, the Washout of the subject is also predicted. An evaluation is then performed collectively upon the environment data, the subject data, the perceived subject data and Washout timing, block 608.

It is to be understood and appreciated that Washout, while important for some illusions, may be a non-factor for others such as the Leans illusion where it is the attitude changes below human threshold levels that are at issue. In such instances, the evaluation of Washout as effectively zero does not alter the prediction, as the environment data indicate the subtle onset of time sequence events that will identify the SD event.

More specifically, in at least one embodiment, the above models of the Leans, Coriolis and Graveyard Spiral illusions are employed. If the collected data indicate a probability for an SD event under any of these models, decision 610, the method directs that countermeasure actions be taken. Of course, it is entirely within reason that the probability of an SD event is effectively zero. In such a case, the data are recorded for later use and post hoc analysis, block 612 (including at least environmental data and subject data). If the operations with the environment are continuing (e.g., still in flight), the method continues with the collection of data, returning to block 600.

If, as in decision 610, there is a probability of an SD event, the degree of probability, i.e., the certainty of the event, is evaluated in decision 614. Where the probability is low, a cautionary countermeasure is implemented, block 616. Such a cautionary countermeasure may include a warning light, auditory warning or other action selected to help inform the subject that an SD event may be occurring. Where the probability is high, decision 616, a more intense countermeasure is initiated, such as an emergency countermeasure, block 618.

With respect to the evaluation of data, block 608, the collection of external world data in at least one embodiment advantageously improves calculation of the probability of an SD event. More specifically, in a setting where visibility is clear and the subject has visual awareness of the geography around him or her, and from that awareness a perception of his or her attitude and orientation, the vestibular illusions may be significantly thwarted by the subject's brain. The predictive method, such as that embodied by SD system 100 may therefore lower the prediction of an SD event in light of the external world data.

However, where visibility is limited and it is not possible to visually discern the surrounding geography, such as an when flying during overcast conditions or in haze, the predictive method, such as that embodied by SD system 100 may raise the prediction of an SD event in light of the external world data.

By predicting not only the probability of an SD event but also the type of SD event, the choice of countermeasure implemented to combat the SD event is advantageously improved. For example, if a subject is experiencing a Coriolis illusion, a blinking light or auditory alert will likely be of little value to the subject or the environment, whereas engaging autopilot or ejecting the subject may save either or both of the pilot and the aircraft. In addition, warning lights as countermeasures may have diminished effectiveness in a bright setting, just as auditory alerts may be diminished in loud settings. Likewise, in an increased G setting, tactile countermeasures may be masked or overshadowed by the forces already at play upon the pilot's body, thus making a strobe light or audio countermeasure more effective. The selectivity of different countermeasures, as well as the degree of the countermeasure (e.g., unique display symbol, blinking light to flashing strobe, audio warning to alarm siren to changing the pitch of a continuous artificial wind sound, activation of a warning signal to flashing messages across multiple displays, tactile sensations of temperature to blasts of cold air, tactile sensations of pressure or vibration (such as from a vest or seat) to electrical shock, mild to extreme odors, engaging auto-pilot or auto-recovery to automated ejection of the subject, recorded verbal requests to recorded verbal orders, etc . . . ) permits the system to administer the most effective countermeasure or countermeasures so as to effectively combat the SD event.

In at least one embodiment, subject workload data are also received from the subject. Workload, as in what the subject is doing, may increase or decrease the subject's susceptibility to SD events and may alter the effectiveness of certain countermeasures. Human beings are capable of performing multiple tasks simultaneously; this ability is otherwise known as parallel processing.

In parallel processing, human beings use sensory channels, processing resources, and response channels (somatic, auditory, visual, vestibular, olfactory, psychomotor-primary, psychomotor-secondary and cognitive) used to greater or lesser degrees for different tasks. If the visual channels and auditory channels are in high use, such as when approaching for landing and communicating with the control tower, the subject pilot will likely respond more quickly to countermeasures administered to less involved channels, such as the olfactory or tactile. Moreover, in an embodiment utilizing subject workload data, countermeasures are further selected for non-taxed workload channels.

Whether based on external world data, workload data or combinations thereof, selectively choosing from a panel of countermeasures is advantageous. Selectively choosing from a panel of countermeasures including, but not limited to, auditory, visual, olfactory, tactile and mechanical intervention provides a significant advantage in providing at least one countermeasure with the highest likelihood of being acknowledged and acted upon by the subject in combating SD.

Following the implementation of a countermeasure, the data (including at least environmental data and subject data) are recorded for later use and post hoc analysis, block 612. In at least one alternative embodiment, the perceived subject attitude is also recorded. If the operations with the environment are continuing (e.g., still in flight), the method continues with the collection of data, returning to block 600. Such a return further aids in evaluating the effectiveness of the implemented countermeasure, blocks 616, 618. If the collected data again indicate the probability of an SD event, decision 610, the countermeasure is continued, increased or augmented with additional countermeasures.

The analysis capabilities of the SD system 100 provide significant advancement in understanding SD events and devising further methods and systems to overcome them. For example, flight safety researchers can utilize the SD system 100 not only in accident reconstruction, but also to identify situations that might tend to induce or increase the opportunity for SD events to occur. FIG. 7 illustrates a high level embodiment of this post hoc review.

In order to act in a post hoc manner, environment data and subject data from a subject within the environment must be pre-recorded. As indicated in blocks 700, 702, these data are collected by the SD system 100. Moreover, the stream of data may be read into a memory array, accessed sequentially or otherwise made available to the input routine 102 of SD system 100 as is appropriate for the physical embodiment of SD system 100 and the volume of data provided. In at least one embodiment, the subject Threshold values and Washout values, as well as the illusion model parameters, are also collected so as to enhance tailoring the post hoc review to a particular subject and or set of conditions, as indicated by optional block 704.

To provide a sequence to the events, in at least one embodiment, the environment data are received as a series of distinct data elements, such as time-stamped data packets. Commencing with the first environment data element, the perceived subject attitude is calculated for the environment data element, block 706.

In accordance with at least the above models (Leans, Coriolis and Graveyard Spiral illusion), the environment data, subject data and perceived subject attitude are evaluated to determine the probability of an SD event, block 708. The probability as calculated is then reported to the SD system user performing the post hoc review, block 710. In at least one embodiment, such a report is made via a display screen upon which the environment data, subject data, perceived subject attitude and probability of an SD event are graphically represented.

If the end of the environment data has been reached, decision 712, the post hoc analysis process ends. If more elements of environment data remain, the process increments to the next element of environment data, block 714, and re-calculates the perceived subject attitude, block 706.

In at least one embodiment, the post hoc review process further includes a review of which countermeasures were initiated when the SD event was evaluated in real time, thus permitting the post hoc reviewer to evaluate the effectiveness of the countermeasures.

This post hoc review advantageously permits a researcher to identify similarities and differences between the collected data and similar pre-recorded event data. For example, flight patterns over certain geographic regions, or occurring at certain times of day or in certain types of weather may be identified as posing a greater risk of SD events to pilots. In at least one embodiment, this comparison review process may be incorporated as an automated component of the SD system 100. In other words, when evaluating environment data, subject data and perceived attitude data in real time, an enhanced SD system 100 may also review similar data from pre-recorded events to further enhance the prediction of an SD event.

Changes may be made in the above methods, systems and structures without departing from the scope hereof. It should thus be noted that the matter contained in the above description and/or shown in the accompanying drawings should be interpreted as illustrative and not in a limiting sense. The following claims are intended to cover all generic and specific features described herein, as well as all statements of the scope of the present method, system and structure, which, as a matter of language, might be said to fall therebetween.

What is claimed is:

1. A computer-readable medium on which is stored a computer program for detecting, analyzing and responding to a spatial disorientation event, the computer program comprising:
an input routine operatively associated with an input device for receiving real time data, recorded data, subject preference information or combinations thereof, the data including:
environment data from an environment, the environment data including true position and orientation of the environment; and
subject data from a subject within the environment;
a vestibular attitude calculator routine for computing the perceived subject attitude of the subject within the environment based on the environment data and subject data, the vestibular attitude calculator including:
a Washout routine to calculate a Washout value;
a vestibular illusion routine to calculate a probability of a vestibular illusion; and
a Threshold adjustment routine permitting adjustment of Washout thresholds and vestibular thresholds based on provided subject preference information;
a countermeasure routine operating in response to the Washout value and the probability of the vestibular illusion; and
an output routine operatively associated with an output device to provide the true position and orientation of the environment and the perceived subject attitude.

2. The computer-readable medium of claim 1, the Washout value being calculated as a non-linear element.

3. The computer-readable medium of claim 1, the input routine further including external world data.

4. The computer-readable medium of claim 1, wherein the countermeasure routine is operatively associated with a plurality of countermeasure devices, the choice and activation of a countermeasure determined by the Washout value, the probability of the vestibular illusion, and combinations thereof.

5. The computer-readable medium of claim 4, wherein the choice and activation of the countermeasure is further determined by the environment data.

6. The computer-readable medium of claim 1, wherein the computer program is executed to perform a post-hoc analysis of the subject within the environment.

7. The computer-readable medium of claim 1, wherein the environment is an aircraft in flight and the subject is a pilot.

8. The computer-readable medium of claim 1, wherein the vestibular attitude calculator further includes a workload calculator routine for calculating the workload of the subject within the environment.

9. The computer-readable medium of claim 1, wherein the subject data are recorded from at least one device worn by the subject.

10. The computer-readable medium of claim 1, wherein the subject data are recorded from one or more environment controls operable by the subject.

11. The computer-readable medium of claim 1, wherein the vestibular attitude calculator routine identifies the spatial disorientation event as either a somatogravic illusion or a somatogyral illusion.

12. The computer-readable medium of claim 11, wherein the somatogyral illusion is further identified as a Leans illusion, a Coriolis illusion or a Graveyard Spiral illusion.

13. The computer-readable medium of claim 11, wherein the countermeasure routine further operates in response to the identified spatial disorientation event.

14. A method for analyzing a spatial disorientation event post-hoc, comprising:
collecting environment data elements recorded from an environment;
collecting subject data recorded from a subject within the environment;
calculating perceived subject attitude of the subject within the environment for each environment data element;
evaluating the environment data, the subject data and the perceived subject attitude to determine a probability of a spatial disorientation event; and
reporting the probability of the spatial disorientation event.

15. The method of claim 14, wherein the subject data are collected from at least one device worn by the subject.

16. The method of claim 14, wherein the subject data are collected from at least one environment control operable by the subject.

17. The method of claim 14, wherein the spatial disorientation event is identified as a vestibular illusion.

18. The method of claim 17, wherein the vestibular illusion is further identified as, a Leans illusion, a Coriolis illusion, or a Graveyard Spiral illusion.

19. The method of claim 14, wherein the environmental data are recorded at predetermined time intervals, the perceived subject attitude calculated for each time interval.

20. The method of claim 14, wherein the environment is an aircraft in flight and the subject is a pilot.

21. The method of claim 14, wherein post-hoc analysis includes determining any similarities or differences between the collected data and pre-recorded event data.

22. The method of claim 14, wherein the method is stored on a computer-readable medium as a computer program, which when executed by a computer will perform the steps of post-hoc spatial disorientation analysis.

23. A method for combating spatial disorientation, comprising:
collecting real time environment data from an environment, the environment data including true position and orientation of the environment;
collecting real time subject data from a subject within the environment;
calculating perceived subject attitude of the subject within the environment for one or more environment data elements and predicting Washout;
evaluating the environment data, the subject data, the perceived subject attitude and Washout to determine the probability of a spatial disorientation event and a type of spatial disorientation event;
implementing, in response to the probability of a spatial disorientation event, at least one countermeasure, the countermeasure selectively chosen from a group of multi sensory countermeasures and countermeasure actions based on the environment data and spatial disorientation probability; and
recording the environmental data and subject data as event data for post-hoc review.

24. The method of claim 23, wherein the Washout is evaluated as a non-linear element.

25. The method of claim 23, further including collecting external world data, wherein evaluating includes evaluating the external world data to determine the probability of a spatial disorientation event.

26. The method of claim 23, wherein determining the probability of the spatial disorientation event includes identifying the spatial disorientation event as a vestibular illusion.

27. The method of claim 26, wherein the vestibular illusion is further identified as a Leans illusion, a Coriolis illusion, or a Graveyard Spiral illusion.

28. The method of claim 23, wherein the environment data are received from a first location within the environment and the subject data are received from a second location within the environment, the second location being different from the first location.

29. The method of claim 23, wherein the group of countermeasures includes auditory, visual, olfactory, tactile, auto-recovery, auto-ejection and combinations thereof.

30. The method of claim 23, further including receiving subject workload data from the subject within the environment, wherein implementing comprises implementing a countermeasure selected for a non-taxed workload channel.

31. The method of claim 23, wherein as the probability of a spatial disorientation event increases, the implemented countermeasure increases from a cautionary to an emergency countermeasure.

32. The method of claim 23, wherein the event data are stored with similar pre-recorded event data, post hoc review including determining similarities or differences between the event data and the pre-recorded event data.

33. The method of claim 23, wherein the method is stored on a computer-readable medium as a computer program, which when executed by a computer will perform the steps of real time and post-hoc spatial disorientation analysis.

34. A method for combating spatial disorientation, comprising:
collecting real time environment data from an environment, the environment data including true position and orientation of the environment;
collecting real time subject data from a subject within the environment;
comparing the collected data with pre-recorded event data to determine any similarities or differences between the event data and the pre-recorded event data and to predict subject response under similar environmental conditions;
calculating perceived subject attitude of the subject within the environment for one or more environment data elements and predicting Washout, the calculations including the predicted subject response;
evaluating the environment data, the subject data, the perceived subject attitude and Washout to determine a probability of a spatial disorientation event and a type of spatial disorientation event;
implementing, in response to the probability of a spatial disorientation event, at least one countermeasure, the countermeasure selectively chosen from a group of multi sensory countermeasures and countermeasure actions based on the environment data and the spatial disorientation probability; and
recording the environmental data and subject data as event data for post-hoc review.

35. The method of claim 34, wherein the Washout is evaluated as a non-linear element.

36. The method of claim 34, wherein determining the probability of the spatial disorientation event includes identifying the spatial disorientation event as a vestibular illusion.

37. The spatial method of claim 36, wherein the vestibular illusion is further identified as a Leans illusion, a Coriolis illusion, or a Graveyard Spiral illusion.

38. The method of claim 34, further including receiving subject workload data from the subject within the environment, the implemented countermeasure further selected for a non-taxed workload channel.
